(19) United States
(12) Patent Application Publication
(10) Pub. No.: US 2024/0147987 A1
(43) Pub. Date: May 9, 2024

(54) METHOD FOR IMPROVING THE EFFECT OF THE OOCYTE CRYOPRESERVATION BY REDUCING THE MITOCHONDRIAL TEMPERATURE

(71) Applicants: China Agricultural University, Beijing (CN); The First Hospital of Hebei Medical University, Shijiazhuang (CN)

(72) Inventors: Xiangwei FU, Beijing (CN); Jun LI, Shijiazhuang (CN); Dan ZHOU, Beijing (CN); Guizhen ZHOU, Beijing (CN); Yunpeng HOU, Beijing (CN)

(73) Assignees: China Agricultural University, Beijing (CN); The First Hospital of Hebei Medical University, Shijiazhuang (CN)

(21) Appl. No.: 18/382,121

(22) Filed: Oct. 20, 2023

(30) Foreign Application Priority Data
Nov. 4, 2022 (CN) 202211377740.8

(51) Int. Cl. A01N 1/02; C12N 5/075

(52) U.S. Cl. CPC A01N 1/0284; A01N 1/0221; C12N 5/0609; C12N 2500/33; C12N 2500/34

(57) ABSTRACT

A method for improving the effect of the oocyte cryopreservation by reducing the mitochondrial temperature is provided. The invention uses a culture medium containing metformin for pretreatment before oocyte freezing; the pre-treated oocytes are then placed in a 20% ethylene glycol solution for balancing, and then the oocytes are placed in a vitrification solution for freezing and are submerged into liquid nitrogen. The method of this invention can effectively reduce the mitochondrial temperature of porcine oocytes, reduce the fluidity of cell membrane, and does not affect the embryonic development after parthenogenetic activation, it can restore the mitochondrial temperature of oocytes after thawing, improve the survival rate after thawing, which can effectively improve the utilization rate of frozen oocytes, it has wide application prospect.

### CROSS REFERENCE TO THE RELATED APPLICATIONS

This application is based upon and claims priority to Chinese Patent Application No. 202211377740.8, filed on Nov. 4, 2022, the entire contents of which are incorporated herein by reference.

### TECHNICAL FIELD

The invention belongs to the field of oocyte cryopreservation technology, especially relates to a method for improving the effect of the oocyte cryopreservation by reducing the mitochondrial temperature.

### BACKGROUND

Cryopreservation of porcine mature oocytes is of great significance for the establishment of germplasm resources and biomedical research. At present, the survival rate of cryopreserved porcine oocytes is low, and the cryopreservation efficiency of different laboratories varies greatly (10%-70%). The cryopreservation efficiency cannot meet the production needs, and it is still in the scientific research stage. It is urgent to develop an effective cryopreservation method.

In recent years, it has been found that a decrease in the freezing resistance of oocytes can be caused by a large amount of deposition of lipid droplets (LDS, fat exists in the form of particles). For example, the average fat content in porcine oocytes is 161 ng, which is nearly 2 times that of sheep oocytes (89 ng), 3 times that of bovine oocytes (63 ng), and 40 times that of mouse (4 ng) and human oocytes. In homeothermal animals, part of the energy produced by the cell's mitochondria is involved in cell metabolism in the form of ATP, and the other part is released in the form of heat energy to maintain the cell's temperature. When the mitochondrial respiratory chain is under normal operation, the temperature of mitochondria can reach about 50° C., which also means that the damage to mitochondria is far greater than that of other organelles when using ultra-low temperature cryopreservation technology.

Metformin is the first-line medicine for type 2 diabetes. It can maintain glucose homeostasis by increasing glucose uptake in peripheral tissues, such as the liver and skeletal muscle. Therefore, it is widely used in the treatment of insulin resistance and type 2 diabetes. In vivo experiments indicated that metformin could alleviate the overweight, high glucose, high fat, and glucose intolerance of high-fat diet mice. The effect of metformin on oocyte mitochondrial temperature and its application in cryopreservation technology have not been reported.

### SUMMARY

In view of the shortcomings of the existing technology, the invention provides a compound that can effectively reduce the mitochondrial temperature, the invention evaluates the role of the compound for reducing the damage of mitochondria caused by severe temperature changes during freezing and thawing, thereby improving the quality of vitrified oocytes.

In order to achieve the above purpose, the invention provides the following solution:

One of the purposes of the invention is to provide a method for improving the effect of the oocyte cryopreservation by reducing the mitochondrial temperature, including the following steps:
(1) pretreating oocytes with a culture medium containing metformin before cryopreservation;
(2) placing the pretreated oocytes in 20% ethylene glycol (EG) solution for equilibrium treatment, and then placing it in a vitrification solution for freezing, and then placing the oocytes in the front end of the Cryotop carrier, each Cryotop can freeze 10-20 oocytes (adjusted according to the proficiency of the operator), and putting the carrier into liquid nitrogen for cryopreservation.

Furthermore, the preparation procedure includes the following steps:
collecting Cumulus-oocyte complexes (COCs) from follicles with a diameter of 3-8 mm on the surface of the ovary, washing three times with TL-HEPES-0.3% bovine serum albumin (BSA) solution, and oocytes are matured in the in vitro maturation fluid for 42-44 h, then matured COCs are digested in H199 containing 0.1% hyaluronidase and granulosa cells are removed to obtain MH oocytes;
among them, the in vitro maturation fluid is a TCM-199 fluid containing 2.78 mM D-glucose, 0.91 mM sodium pyruvate, 0.57 mM cysteine, 50 mg/mL streptomycin, 75 mg/mL penicillin, 10% porcine follicular fluid (from porcine ovarian follicles), 0.01 U/mL follicle stimulating hormone (FSH), 0.01 U/mL luteinizing hormone (LH), and 10 ng/mL epidermal growth factor (EGF);
H199 solution is a TCM-199 solution containing 0.01 M 4-hydroxyethyl piperazine ethanesulfonic acid (HEPES), 0.01 M 4-hydroxyethyl piperazine ethanesulfonic acid sodium salt (HEPES-NA), 5 mM NaHCO₃, 50 mg/mL streptomycin, and 65 mg/mL penicillin.

Furthermore, the culture medium described in step (1) is a Tyrode's lactate (TL)-HEPES-0.3% BSA solution;
the TL-HEPES-0.3% BSA solution contains 6.662 g/L NaCl, 0.238 g/L KCl, 0.168 g/L NaHCO₃, 0.046 g/L KH₂PO₄, 2.383 g/L HEPES, 0.022 g/L sodium pyruvate, 1.121 g/L sodium lactate, 2.186 g/L sorbitol, 0.01 g/L streptomycin, 0.05 g/L penicillin, 0.102 g/L MgCl₂-6H₂O, 0.294 g/L CaCl₂·2H₂O and 3 g/L BSA.

Furthermore, the culture medium contains 400 μM metformin.

Furthermore, the pretreatment duration described in step (1) is 1 h.

Furthermore, the balance treatment duration described in step (2) is 3 min.

Furthermore, the vitrification solution described in step (2) is EDFS40 (DPBS with 12% FBS, 0.3 mol/L sucrose, 18% Ficoll, 40% EG), and the cryopreservation duration is 30-40 s.

The beneficial effects of the invention are as follows.

The invention reduces the mitochondrial temperature by metformin, thereby improving the oocyte freezing effect, where metformin can significantly reduce the oocyte mitochondrial temperature and cell membrane fluidity without affecting the oocyte development ability. Pretreatment with metformin before cryopreservation can reduce the volume of lipid droplets in vitrified oocytes, which is benefit for the recovery of mitochondrial temperature and improves the survival rate after thawing, thus effectively improves the utilization rate of frozen oocytes. It is of great significance and provides wide application prospects for improving the efficiency of oocyte cryopreservation.

### BRIEF DESCRIPTION OF THE DRAWINGS

The application file contains at least one drawing executed in color. Copies of this patent or patent application publication with color drawing(s) will be provided by the Office upon request and payment of the necessary fee.

In order to more clearly explain the embodiment of the invention or the technical solution in the existing technology, a brief introduction to the drawings needed in the embodiment is shown in the following. Obviously, the drawings in the following description are only some embodiments of the invention. For ordinary technicians in this field, other drawings can be obtained according to these drawings without paying creative labor.

FIGS. 1A-l B are effects of different concentrations of metformin on the mitochondrial temperature of porcine oocytes, FIG. 1A is a mitochondrial temperature probe MTY fluorescence staining diagram, and FIG. 1B is a comparison of fluorescence intensity;

FIGS. 2A-2C are effects of metformin on the development of porcine oocytes, FIG. 2A is a development diagram of oocyte maturation and parthenogenetic embryo, FIG. 2B and FIG. 2C are the comparisons of cleavage rate and blastocyst development rate between the control group and the metformin treatment group;

FIGS. 3A-3B are effects of metformin treatment on plasma membrane fluidity of porcine oocytes, FIG. 3A is a recovery of fluorescence at different times after plasma membrane bleaching, and FIG. 3B is a change of fluorescence intensity with time after plasma membrane bleaching;

FIGS. 4A-4C are effects of metformin on the survival of porcine frozen oocytes, FIG. 4A is an FDA staining of fresh oocytes; FIG. 4B is an FDA staining of conventional vitrified oocytes; FIG. 4C is an FDA staining of vitrified oocytes after metformin pretreatment;

FIGS. 5A-5B are effects of metformin on the mitochondrial temperature of vitrified oocytes, FIG. 5A is a fluorescence staining of MTY probe in vitrified oocytes. FIG. 5B is a comparison of MTY fluorescence intensity among the control group, freezing group, and metformin pretreatment freezing group;

FIGS. 6A-6C are effects of metformin on the ultrastructure of lipid droplets in vitrified oocytes; FIG. 6A is an ultrastructure of fresh oocytes; FIG. 6B is an ultrastructure of conventional vitrified oocytes; FIG. 6C is an ultrastructure of vitrified oocytes after metformin pretreatment;

FIGS. 7A-7B are effects of rotenone treatment at different concentrations on the mitochondrial temperature of porcine oocytes; FIG. 7A is a MTY fluorescence staining of oocytes treated with rotenone at different concentrations; FIG. 7B is a comparison of MTY fluorescence intensity of oocytes in different groups;

FIGS. 8A-8C are effects of rotenone treatment on the development of porcine oocytes, FIG. 8A is a picture of in vitro maturation, cleavage, and blastocyst formed after the parthenogenetic activation of oocytes in different groups. FIG. 8B, FIG. 8C are the comparisons for cleavage rate and blastocyst rate of the control group and rotenone treatment group;

FIGS. 9A-9B are effects of different concentrations of oligomycin on the mitochondrial temperature of porcine oocytes; FIG. 9A is an MTY staining of oocytes treated with different concentrations of oligomycin; FIG. 9B is a comparison of MTY fluorescence intensity of oocytes in different groups;

FIGS. 10A-10C are effects of different concentrations of oligomycin treatment on the development of porcine oocytes; FIG. 10A is a diagram of oocyte maturation, cleavage, and blastocyst development treated with different concentrations of oligomycin. FIG. 10B and FIG. 10C are the comparisons of cleavage rate and blastocyst rate in different treatment groups.

FIGS. 11A-11C are effects of oligomycin treatment on the survival of porcine frozen oocytes, FIG. 11A is an FDA staining of fresh oocytes; FIG. 11B is an FDA staining of conventional vitrified oocytes; FIG. 11C is an FDA staining of vitrified oocytes after oligomycin treatment;

FIGS. 12A-12B are effects of different concentrations of UK5099 on the mitochondrial temperature of porcine oocytes, FIG. 12A is an MTY staining of oocytes treated with different concentrations of UK5099, FIG. 12B is a comparison of MTY fluorescence intensity of oocytes in different groups;

FIGS. 13A-13C are effects of different concentrations of UK5099 treatment on the development of porcine oocytes, FIG. 13A is a diagram of oocyte maturation, cleavage, and blastocyst development treated with different concentrations of UK5099, FIG. 13B and FIG. 13C are the comparisons of cleavage rate and blastocyst rate in different treatment groups.

FIGS. 14A-14C is an effect of UK5099 treatment on the survival of porcine frozen oocytes, and FIG. 14A is an FDA staining of fresh oocytes, FIG. 14B is an FDA staining of conventional vitrified oocytes, FIG. 14C is an FDA staining of vitrified oocytes after pretreatment with UK5099.

### DETAILED DESCRIPTION OF THE EMBODIMENTS

In order to make the technical personnel in this field better understand the technical solution of the invention, the invention is further described in detail with the implementation example.

In the following embodiments, the preparation process of oocytes is:

Fresh ovaries (all from young sows) were collected from the slaughterhouse and brought back to the laboratory within 2 h in 0.9% (m/v) NaCl solution (containing 75 μg/mL penicillin G and 50 μg/mL streptomycin) at 30-37° C. Cumulus-oocyte complexes (COCs) in follicles with a diameter of 3-8 mm on the surface of ovaries were collected and washed three times with TL-HEPES-0.3% BSA, COCs with more than three layers of cumulus cells and uniform cytoplasm were selected and cultured in four-well plates (Nunc, Denmark, Φ60 mm). The cells were cultured in a carbon dioxide incubator with air containing 5% CO₂, 38.5° C., and saturated humidity (Thermo Electron Corporation, USA) in vitro maturation fluid for 42-44 h to MII stage. Matured COCs were rapidly digested in H199 containing 0.1% hyaluronidase (TCM-199 containing 0.01 M HEPES, 0.01 M HEPES-NA, 5 mM NaHCO₃, 50 mg/mL streptomycin, and 65 mg/mL penicillin), and granulosa cells were removed to obtain the in vitro matured oocytes.

### Example 1

1. Metformin Pretreatment.

The oocytes were pre-treated with 100 μM, 200 μM, 400 μM, and 800 μM metformin for 1 h.

The culture medium was TL-HEPES-0.3% BSA containing 400 μM metformin.
(1) Oocyte Mitochondrial Temperature Detection

100 nM Mito Thermo Yellow dye was added in TL-HEPES-0.3% BSA solution and preheated for 15 min at 37° C. in an incubator containing 5% CO₂, the oocytes were moved into the preheated dye solution in the incubator for 15 min and then washed three times with TL-HEPES-0.3% BSA for 10 min each time, the fluorescence intensity was detected by laser confocal microscopy, and the changes of mitochondrial temperature were analyzed.

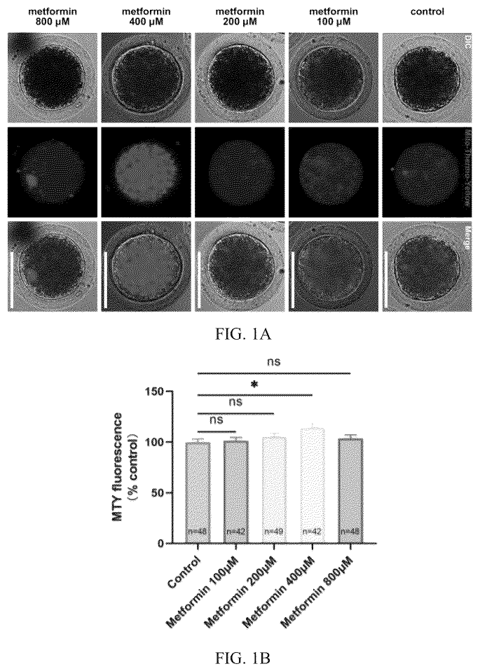

Metformin can effectively reduce mitochondrial heat production. FIGS. 1A-1B showed the effects of different concentrations (100 μM, 200 μM, 400 μM, 800 μM) of metformin treatment for 1 h on mitochondrial temperature, MTY was a specific probe for mitochondrial temperature detection, and its fluorescence intensity was inversely proportional to mitochondrial temperature. As shown in FIGS. 1A-1B, 400 μM metformin treatment for 1 h can significantly reduce mitochondrial temperature (P<0.05).
(2) Parthenogenetic Activation of Oocytes

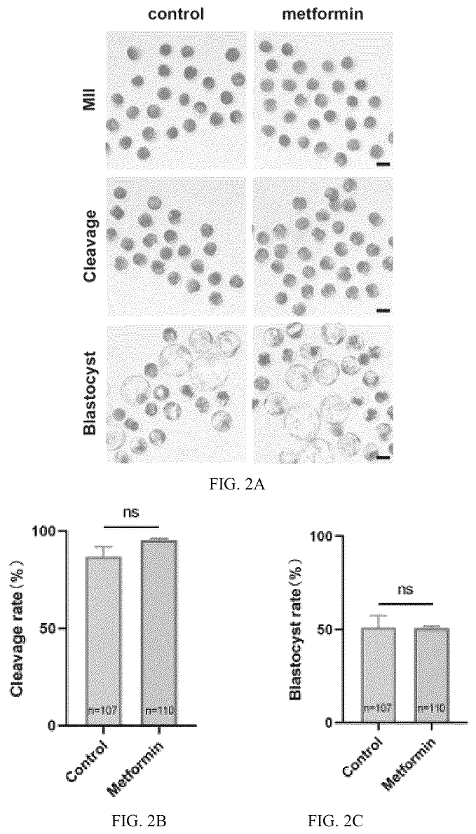

After the first polar body was extruded, mature oocytes with uniform cytoplasm were selected for parthenogenetic activation. The oocytes were washed three times with a pre-heated electro-activation solution, and then the oocytes were placed between electrodes filled with the activation solution, the electro-activation solution was 0.3 M mannitol, 0.05 mM CaCl₂, 0.1 mM MgCl₂, and 0.4% (m/v) bovine serum albumin dissolved in water. An electric fusion meter (Fujihira Industry Co. Ltd, Tokyo, Japan) was used for electrical activation, the electric field intensity was 65 V/mm, the DC pulse duration was 80 μs, and the number of pulses was 1. The activated oocytes were cultured in PZM-3 containing 5 μg/mL cytochalasin B and 10 μg/mL cycloheximide at 38.5° C., 5% CO₂ for 4 h. Then the oocytes were washed and cultured in PZM-3 (day 0). The number of cleavage embryos was counted on day 2 and blastocyst formation was recorded on day 7. As shown in FIGS. 2A-2C, there was no significant difference in cleavage rate and blastocyst development rate between the metformin pretreatment group and the control group, indicating that metformin did not affect embryo development after parthenogenetic activation.
(3) Oocyte Membrane Fluidity Detection

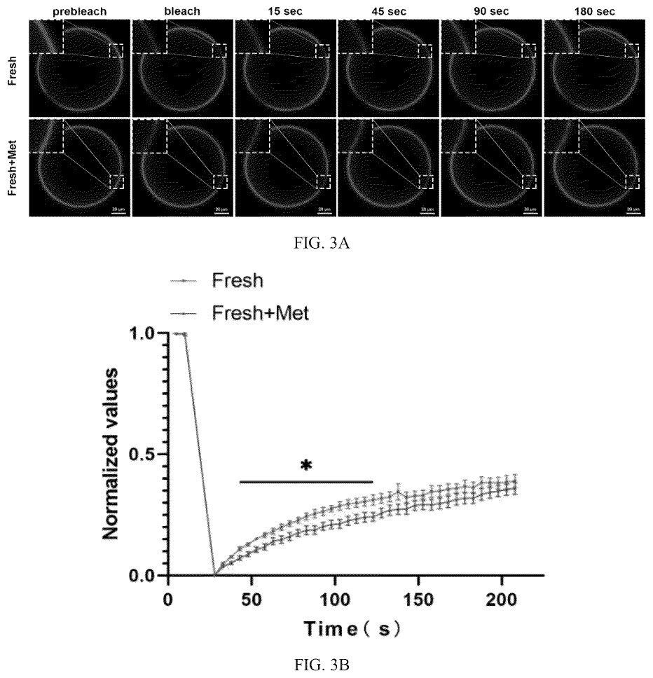

The membrane fluidity of oocytes was detected by fluorescence recovery after photobleaching (FRAP). The oocytes were first placed in TL-HEPES-0.3% BSA solution containing cell membrane fluorescent probe Dil, and incubated at 37° C. in the incubator containing 5% CO₂ for 10 min. After incubation, the oocytes were washed three times within TL-HEPES-0.3% BSA solution, and then the oocytes were placed in a confocal dish. FRAP experiments were performed on a confocal microscope. Firstly, a bleaching area was selected, and three fluorescence images were collected before bleaching, with an interval of 5 s for each image. The bleaching fluorescence intensity was 100%, and the bleaching time was 7.93 s. Recovery time was 3 min after bleaching, and fluorescence images were collected at 5 s intervals. The fluorescence intensity of the bleaching area was statistically analyzed and the data were normalized. As shown in FIGS. 3A-3B, metformin pretreatment significantly decreased its fluidity (P<0.05).

2. Freezing and Thawing.

The room temperature was adjusted to 25±1° C. so that the test apparatus and reagents were fully balanced, the experiment was carried out on a 37° C. thermostat, the oocytes were transferred into 20% EG solution for 3 min, and then transferred into the vitrification solution EDFS40 for 30-40 s, the oocytes were then placed on the cryocarrier Cryotop. Each Cryotop can freeze 10-20 oocytes (adjusted according to the operator's proficiency), and then it was directly submerged into liquid nitrogen (the whole process does not exceed 1 min). When thawing the oocytes, the Cryotop was taken out from liquid nitrogen, and the oocytes were immediately immersed in PBS containing 1M sucrose (containing 20% FBS) on a thermostatic table (37° C.) for 1 min, and then transferred to PBS containing 0.5M and 0.25M sucrose (containing 20% FBS) for 3 min, and then placed in PBS containing 20% FBS for recovery for 5 min, and then washed three times with mature medium and placed in mature medium for recovery for 2 hours.
(1) Survival Rate Detection of Oocytes after Thawing

The survival rate of oocytes was detected by FDA (fluorescein diacetate) staining. The collected oocytes were incubated in TL-HEPES-0.3% BSA solution containing 2.5 μg/mL FDA for 1 min and then washed three times with TL-HEPES-0.3% BSA solution, the oocytes were placed on a petri dish and observed using a fluorescence microscope, the oocytes with green fluorescence were considered to be alive.

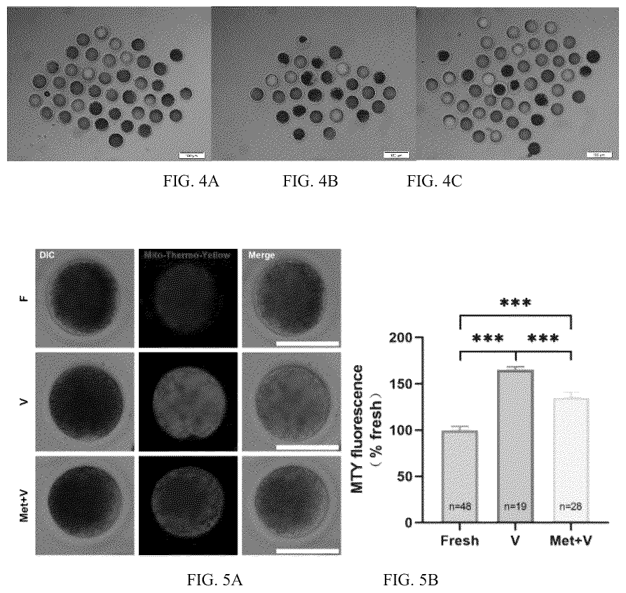

FDA staining was used to determine the survival rate of vitrified oocytes. As shown in FIGS. 4A-4C and Table 1, the survival rate of frozen oocytes pretreated with 400 μM metformin for 1 h was significantly higher than that of the traditional freezing group (81.36% vs.71.85%, P<0.05).

TABLE 1
Effect of metformin pretreatment on the survival
rate of vitrified porcine oocytes

| | | | Metformin |
|---|---|---|---|
| | | Freezing | pretreatment |
| Group | Fresh group | group | freezing group |
| Number of | 149 | 238 | 220 |
| oocytes (n) | | | |
| Survival | 100ª(149/149) | 71.85^{b}(171/238) | 81.36^{c}(179/330) |
| rate (%) | | | |

(2) Effect of Metformin on the Mitochondrial Temperature of Vitrified Oocytes

The effect of metformin pretreatment on the mitochondrial temperature of vitrified oocytes was further analyzed. As shown in FIGS. 5A-5B, the mitochondrial temperature of conventional frozen oocytes decreased significantly after thawing (P<0.01), while the mitochondrial temperature of the metformin pretreatment group was significantly higher than that of the conventional freezing group (P<0.01).
(3) Ultrastructural Observation of Lipid Droplets in Oocytes

The oocytes were washed with DPBS and fixed overnight at 4° C. in a 0.1M dimethylarsenate buffer containing 2.5% (m/v) glutaraldehyde. After washing with DPBS, oocytes were fixed with 1% osmic acid, then washed with DPBS, and dehydrated in a gradient of 30%, 50%, 70%, 80%, 90%, and 100% acetone. The epoxy resin SPURR was used for embedding-polymerization, and the LKB-V slicing machine (ultra-thin slice thickness 50 nm (diamond knife)) was used for slicing, and then the double staining of uranium acetate-lead citrate was carried out on the copper mesh of the electron microscope, the copper mesh containing the sample was dried in a glass dish overnight, and the image was collected using a transmission electron microscope (TEM) at 80 kv (HITACHI, New Bio-TEM H-7500, Japan).

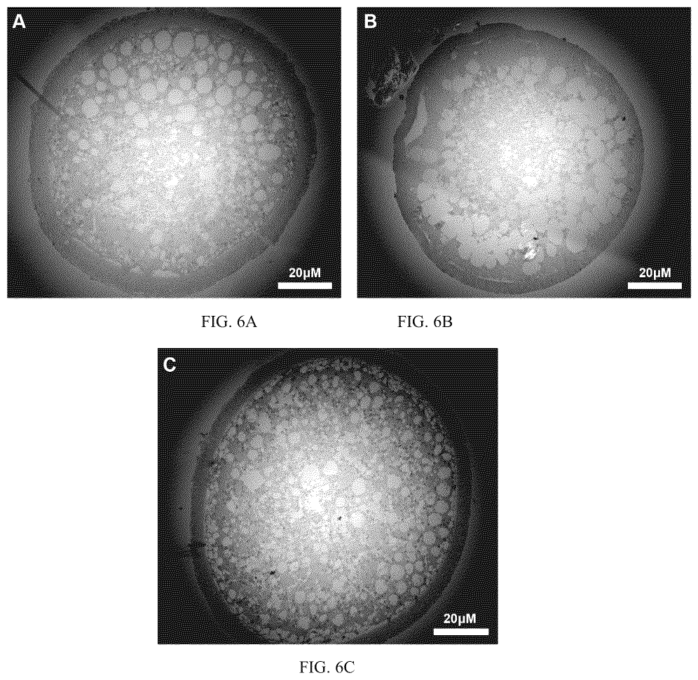

As shown in FIGS. 6A-6C, the volume of lipid droplets in oocytes of the conventional freezing group was huge and the number was big, and the size of lipid droplets in oocytes of the metformin pretreatment group was close to that of the fresh group.

The above results indicate that 400 μM metformin can significantly reduce mitochondrial temperature and reduce the damage to mitochondrial function caused by severe temperature changes during freezing-thawing, which was beneficial to the recovery of mitochondrial temperature and cell survival of oocytes after thawing.

Comparison Case 1

Effects of Rotenone on Mitochondrial Temperature and Development of Porcine Oocytes

The methods of oocyte collection, in vitro maturation, parthenogenetic activation, and mitochondrial temperature detection were the same as those in Example 1. Differently, metformin was changed to different concentrations of rotenone (0.3 μM, 0.6 μM, 1 μM).
(1) Effects of Rotenone on the Mitochondrial Temperature of Porcine Oocytes

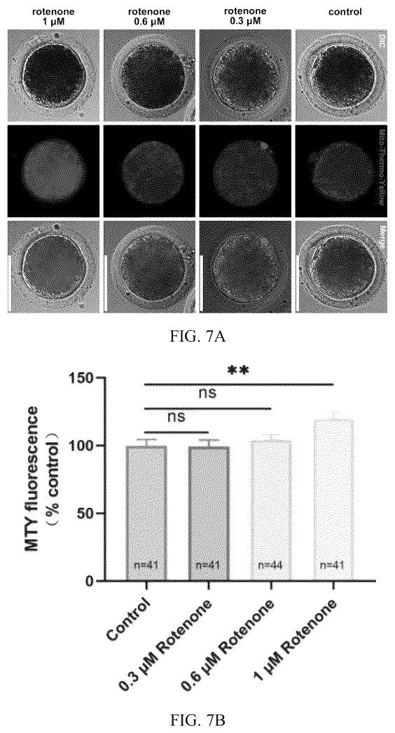

As shown in FIGS. 7A-7B, 1 μM rotenone treatment for 1 h significantly reduced the mitochondrial temperature of oocytes (P<0.01).
(2) Effects of Rotenone on Oocyte Development

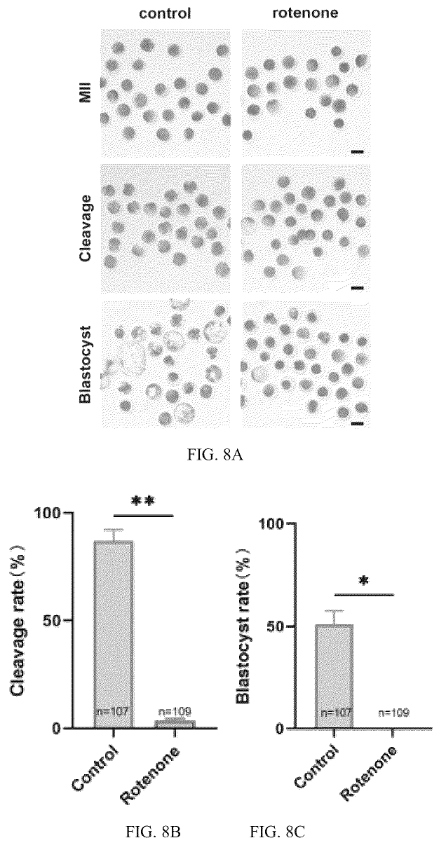

As shown in FIGS. 8A-8C, compared with the control group, the cleavage rate (P<0.01) and blastocyst development rate (P<0.01) of the rotenone-treated group were significantly decreased after parthenogenetic activation.

Comparison Case 2

Effect of Oligomycin on Mitochondrial Temperature of Porcine Oocytes and Its Application in Oocyte Cryopreservation

Oocyte collection, in vitro maturation, mitochondrial temperature detection, parthenogenetic activation, freezing and thawing, and oocyte survival determination are equivalent to Example 1, the difference was that metformin was changed to different concentrations (0.5 M, 1.5 μM, 2.5 μM) of oligomycin treatment.
(1) Effect of Oligomycin on the Mitochondrial Temperature of Porcine Oocytes

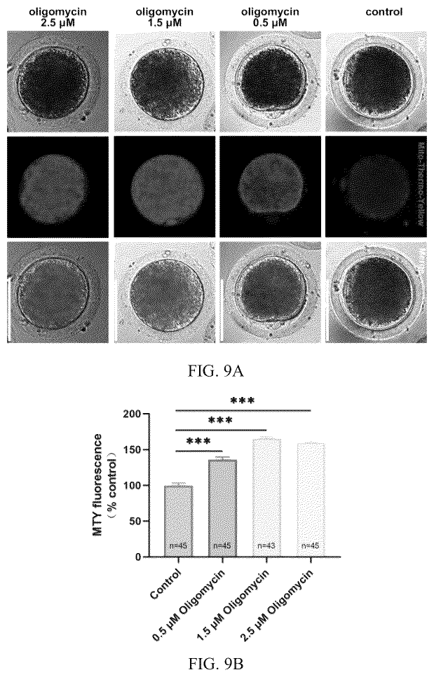

As shown in FIGS. 9A-9B, 0.5 μM, 1.5 μM, and 2.5 μM oligomycin treatment for 1 h can significantly reduce the oocyte mitochondrial temperature (P<0.01).
(2) Effect of Oligomycin on Oocyte Development

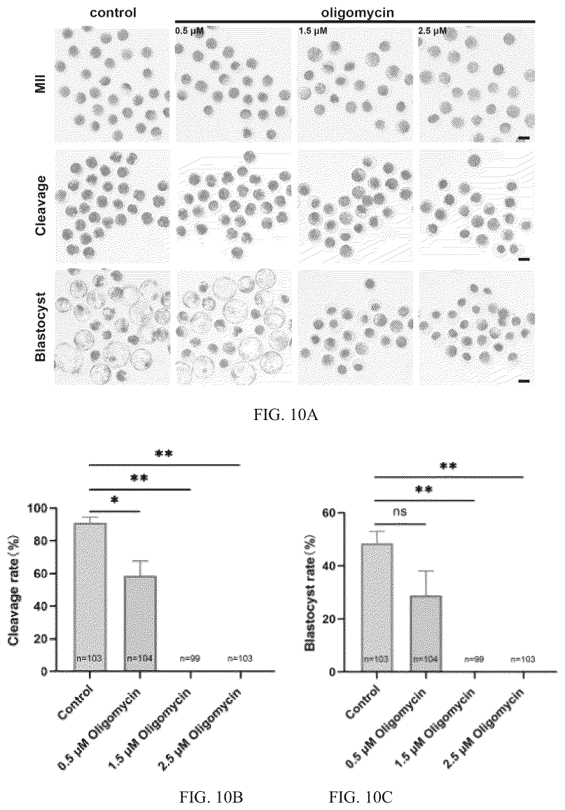

As shown in FIGS. 10A-10C, the cleavage rate of parthenogenetic activation in different concentrations of oligomycin treatment groups was significantly lower than that in the control group (P<0.01). Compared with the control group, the blastocyst development in 1.5 μM (P<0.01) and 2.5 μM (P<0.01) treatment groups was significantly lower than that in the control group, and the blastocyst development rate in 0.5 μM treatment group was also significantly lower than that in the control group.
(3) Effect of Oligomycin Pretreatment on the Survival Rate of Frozen Oocytes

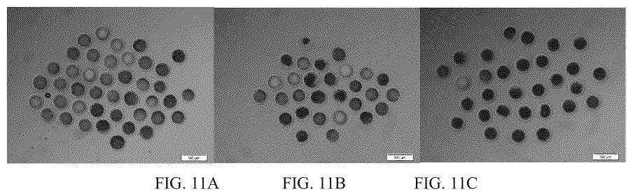

The analysis of oocyte survival rate in the fresh group, freezing group, and oligomycin pretreatment group was shown in FIGS. 11A-11C, as shown in Table 2, the oocyte thawing survival rate in the oligomycin pretreatment group was significantly lower than that in the conventional freezing group (29.79% vs.71.85%, P<0.05).

TABLE 2
Effect of oligomycin pretreatment on
thawing survival of porcine oocytes

| | | | Oligomycin |
|---|---|---|---|
| | | Freezing | pretreatment |
| Group | Fresh group | group | freezing group |
| No of | 149 | 238 | 188 |
| oocytes (n) | | | |
| Survival | 100^{a}(149/149) | 71.85^{b}(171/238) | 29.79^{c}(56/188) |
| rate (%) | | | |

Comparison Case 3

Effect of UK5099 on the mitochondrial temperature of porcine oocytes and its application in oocyte cryopreservation

Oocyte collection, in vitro maturation, oocyte mitochondrial temperature detection, parthenogenetic activation, freezing and thawing, and oocyte survival determination were the same as those in Example 1. Differently, metformin pretreatment was changed to different concentrations (0.5 μM, 1 μM, 2 μM, 4 μM) of UK5099 treatment.
(1) Effect of UK5099 on the Mitochondrial Temperature of Porcine Oocytes

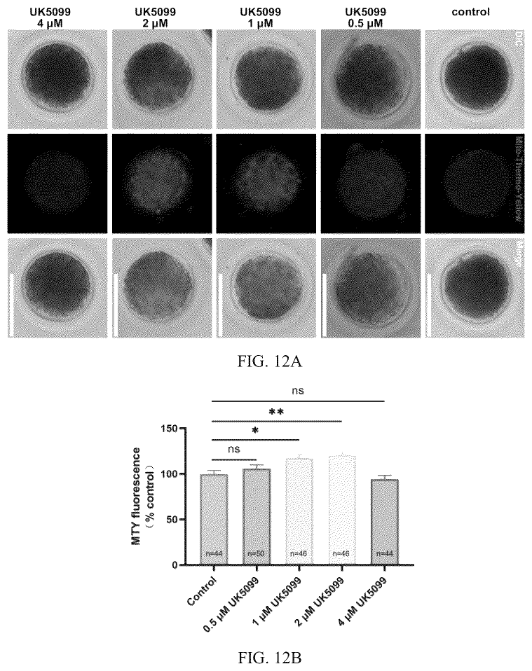

As shown in FIGS. 12A-12B, 1 μM (P<0.05) and 2 μM UK5099 (P<0.01) treatment for 1 h significantly reduced the mitochondrial temperature of oocytes.
(2) The Effect of UK5099 on Oocyte Development

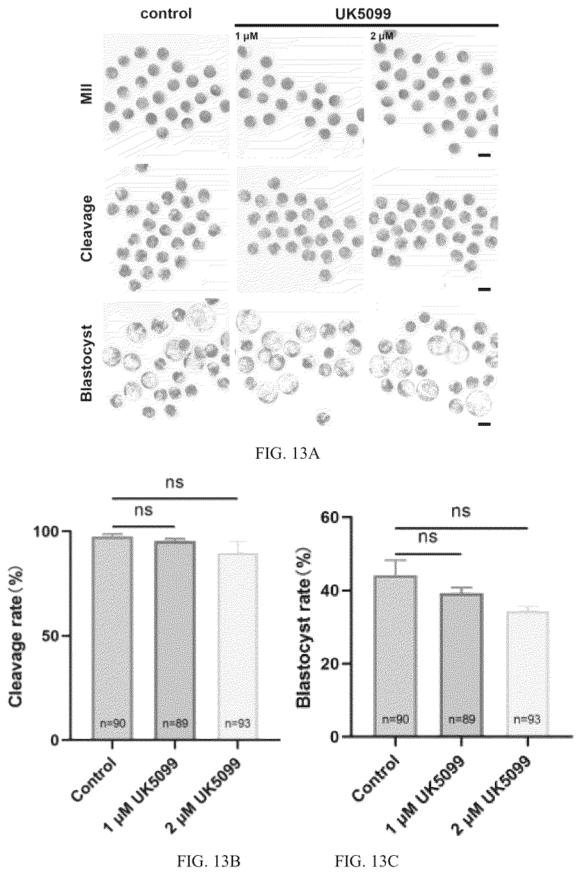

As shown in FIGS. 13A-13C, compared with the control group, the cleavage rate and blastocyst rate after parthenogenetic activation in the 1 μM and 2 μM UK5099 treatment groups were not significantly different from those in the control group, but the cleavage rate and blastocyst rate decreased with the increase of UK5099 concentration.
(3) The effect of UK5099 on the survival of frozen oocytes

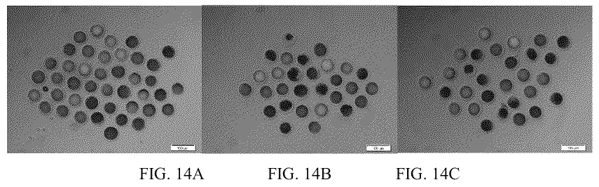

The analysis of oocyte survival rate in the fresh group, freezing group, and UK5099 pretreatment group was shown in FIGS. 14A-14C, as shown in Table 3, there was no significant difference in the oocyte thaw survival rate between UK5099 pretreatment freezing group and conventional freezing group (67.50% vs.71.85%, P>0.05).

TABLE 3
Effects of UK5099 pretreatment on the
survival rate of vitrified porcine oocytes

| | | | UK5099 |
|---|---|---|---|
| | | Freezing | pretreatment |
| Group | Fresh group | group | freezing group |
| Number of | 149 | 238 | 200 |
| oocytes (n) | | | |
| Survival | 100^{a}(149/149) | 71.85^{b}(171/238) | 67.50^{b}(135/200) |
| rate (%) | | | |

In summary, the above results showed that although rotenone, oligomycin, and UK5099 had the biological effects of regulating mitochondrial temperature, rotenone has greater cytotoxicity, and oligomycin can significantly reduce the survival rate of frozen oocytes. UK5099 cannot effectively improve the survival rate of frozen oocytes, while 400 μM metformin can significantly reduce the mitochondrial temperature of porcine oocytes and reduce cell membrane fluidity. Pretreatment with this concentration of metformin before cryopreservation can reduce the volume of lipid droplets in porcine oocytes, restore the mitochondrial temperature of oocytes after thawing, and effectively improve the survival rate of frozen-vitrified oocytes.

The above-mentioned embodiments are only used for describing the preferred method of the invention, which can not be used for limiting the protection scope of the invention. Under the premise of not deviating from the design spirit of the invention, various deformations and improvements made by ordinary technicians in this field to the technical solution of the invention should fall within the protection scope determined by the claims of the invention.

What is claimed is:

1. A method for improving an effect of an oocyte cryopreservation by reducing a mitochondrial temperature, comprising the following steps:
step (1) pretreating an oocyte with a culture medium containing metformin to obtain a pretreated oocyte before the oocyte cryopreservation;
step (2) placing the pretreated oocyte in a 20% ethylene glycol (EG) solution for an equilibrium treatment to obtain an equilibrated oocyte, placing the equilibrated oocyte in a vitrification solution for freezing to obtain a resulting oocyte and placing the resulting oocyte in a front end of a Cryotop carrier, and then placing a carrier containing the resulting oocyte into a liquid nitrogen for the oocyte cryopreservation.

2. The method for improving the effect of the oocyte cryopreservation according to claim 1, wherein the culture medium described in step (1) is a Tyrode's lactate (TL)-4-hydroxyethyl piperazine ethanesulfonic acid (HEPES)-0.3% bovine serum albumin (BSA) solution.

3. The method for improving the effect of the oocyte cryopreservation according to claim 1, wherein the culture medium contains 400 μM metformin.

4. The method for improving the effect of the oocyte cryopreservation according to claim 1, wherein a pretreatment duration of the pretreating described in step (1) is 1 h.

5. The method for improving the effect of the oocyte cryopreservation according to claim 1, wherein a balance treatment duration of the equilibrium treatment described in step (2) is 3 min.

6. The method for improving the effect of the oocyte cryopreservation according to claim 1, wherein the vitrification solution described in step (2) is EDFS40 (DPBS with 12% FBS, 0.3 mol/L sucrose, 18% Ficoll, 40% EG), and a freezing duration of the freezing is 30-40 s.
